SEQUENCE LISTING (22 sequences)

SEQ ID NO: 1 - protein, 594 aa, Artificial Sequence
  The amino acid sequence of the mutated PHA synthase derived from Aeromonas caviae
MSQPSYGPLFEALAHYNDKLLAMAKAQTERTAQALLQTNLDDLGQVLEQGSQQPWQLIQAQMNWWQDQLKLMQHTLLKSAGQPSEPVITPERSDRRFKAEAWSEQPIYDYLKQSYLLTARHLLASVDALEGVPQKSRERLRFFTRQYVSAMAPSNFLATNPELLKLTLESGGQNLVRGLALLAEDLERSADQLNIRLTDESAFELGRDLALTPGRVVQRTELYELIQYSPTTETVGKTPVLIVPPFINKYYIMDMRPQNSLVAWLVAQGQTVFMISWRNPGVAQAQIDLDDYVVDGVIAALDGVEAATGEREVHGIGYCIGGTALSLAMGWLAARRQKQRVRTATLFTTLLDFSQPGELGIFIHEPIIAALEAQNEAKGIMDGRQLAVSFSLLRENSLYWNYYIDSYLKGQSPVAFDLLHWNSDSTNVAGKTHNSLLRRLYLENQLVKGELKIRNTRIDLGKVKTPVLLVSAVDDHIALWQGTWQGMKLFGGEQRFLLAESGHIAGIINPPAANKYGFWHNGAEAESPESWLAGATHQGGSWWPEMMGFIQNRDEGSEPVPARVPEEGLAPAPGHYVKVRLNPVFACPTEEDAA

SEQ ID NO: 2 - protein, 589 aa, Ralstonia eutropha
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 3 - protein, 559 aa, Artificial Sequence
  The amino acid sequence of the mutated PHA synthase derived from Pseudomonas sp.61-3
MSNKNSDDLNRQASENTLGLNPVIGLRGKDLLTSARMVLTQAIKQPIHSVKHVAHFGIELKNVMFGKSKLQPESDDRRFNDPAWSQNPLYKRYLQTYLAWRKELHDWIGNSKLSEQDINRAHFVITLMTEAMAPTNSAANPAAVKRFFETGGKSLLDGLTHLAKDLVNNGGMPSQVDMGAFEVGKSLGTTEGAVVFRNDVLELIQYRPTTEQVHERPLLVVPPQINKFYVFDLSPDKSLARFCLSNNQQTFIVSWRNPTKAQREWGLSTYIDALKEAVDVVSAITGSKDINMLGACSGGITCTALLGHYAALGEKKVNALTLLVTVLDTTLDSQVALFVDEKTLEAAKRHSYQAGVLEGRDMAKVFAWMRPNDLIWNYWVNNYLLGNEPPVFDILFWNNDTTRLPAAFHGDLIEMFKNNPLVRANALEVSGTPIDLKQVTADIYSLAGTNDHITPWKSCYKSAQLFGGKVEFVLSSRGHIRSILNPPGNPKSRYMTSTDMPATANEWQENSTKHTDSWWLHWQAWQAERSGKLKKSPTSLGNKAYPSGEAAPGTYVHER

SEQ ID NO: 4 - DNA, 1785 bp, Artificial Sequence
  The gene encoding the mutated PHA synthase derived from Aeromonas caviae
atgagccaaccatcttatggcccgctgttcgaggccctggcccactacaatgacaagctgctggccatggccaaggcccagacagagcgcaccgcccaggcgctgctgcagaccaatctggacgatctgggccaggtgctggagcagggcagccagcaaccctggcagctgatccaggcccagatgaactggtggcaggatcagctcaagctgatgcagcacaccctgctcaaaagcgcaggccagccgagcgagccggtgatcaccccggagcgcagcgatcgccgcttcaaggccgaggcctggagcgaacaacccatctatgactacctcaagcagtcctacctgctcaccgccaggcacctgctggcctcggtggatgccctggagggcgtcccccagaagagccgggagcggctgcgtttcttcacccgccagtacgtcagcgccatggcccccagcaacttcctggccaccaaccccgagctgctcaagctgaccctggagtccggcggccagaacctggtgcgcggactggccctcttggccgaggatctggagcgcagcgccgatcagctcaacatccgcctgaccgacgaatccgccttcgagctcgggcgggatctggccctgaccccgggccgggtggtgcagcgcaccgagctctatgagctcattcagtacagcccgactaccgagacggtgggcaagacacctgtgctgatagtgccgcccttcatcaacaagtactacatcatggacatgcggccccagaactccctggtcgcctggctggtcgcccagggccagacggtattcatgatctcctggcgcaacccgggcgtggcccaggcccaaatcgatctcgacgactacgtggtggatggcgtcatcgccgccctggacggcgtggaggcggccaccggcgagcgggaggtgcacggcatcggctactgcatcggcggcaccgccctgtcgctcgccatgggctggctggcggcgcggcgccagaagcagcgggtgcgcaccgccaccctgttcactaccctgctggacttctcccagcccggggagcttggcatcttcatccacgagcccatcatagcggcgctcgaggcgcaaaatgaggccaagggcatcatggacgggcgccagctggcggtctccttcagcctgctgcgggagaacagcctctactggaactactacatcgacagctacctcaagggtcagagcccggtggccttcgatctgctgcactggaacagcgacagcaccaatgtggcgggcaagacccacaacagcctgctgcgccgtctctacctggagaaccagctggtgaagggggagctcaagatccgcaacacccgcatcgatctcggcaaggtgaagacccctgtgctgctggtgtcggcggtggacgatcacatcgccctctggcagggcacctggcagggcatgaagctgtttggcggggagcagcgcttcctcctggcggagtccggccacatcgccggcatcatcaacccgccggccgccaacaagtacggcttctggcacaacggggccgaggccgagagcccggagagctggctggcaggggcgacgcaccagggcggctcctggtggcccgagatgatgggctttatccagaaccgtgacgaagggtcagagcccgtccccgcgcgggtcccggaggaagggctggcccccgcccccggccactatgtcaaggtgcggctcaaccccgtgtttgcctgcccaacagaggaggacgccgcatga

SEQ ID NO: 5 - DNA, 260 bp, Artificial Sequence
  The mutated promoter sequence derived from Escherichia coli and the Shine-Dargano sequence derived from Cupriavidus necator H16
caattgtgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacaattgcacgtgcagagagacaatcaaatc

SEQ ID NO: 6 - DNA, 35 bp, Artificial Sequence
  primer
gcgcgcatttaaatctgccaccacgttgtgctcgg

SEQ ID NO: 7 - DNA, 25 bp, Artificial Sequence
  primer
gatttgattgtctctctgccgtcac

SEQ ID NO: 8 - DNA, 40 bp, Artificial Sequence
  primer
ggcagagagacaatcaaatccgcttgcatgagtgccggcg

SEQ ID NO: 9 - DNA, 35 bp, Artificial Sequence
  primer
gcgcgcatttaaattgtcgaccagcttggcatcgc

SEQ ID NO: 10 - DNA, 1770 bp, Ralstonia eutropha
atggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatga

SEQ ID NO: 11 - DNA, 1680 bp, Artificial Sequence
  The gene encoding the mutated PHA synthase derived from Pseudomonas sp.61-3
atgagtaacaagaatagcgatgacttgaatcgtcaagcctcggaaaacaccttggggcttaaccctgtcatcggcctgcgtggaaaagatctgctgacttctgcccgaatggttttaacccaagccatcaaacaacccattcacagcgtcaagcacgtcgcgcattttggcatcgagctgaagaacgtgatgtttggcaaatcgaagctgcaaccggaaagcgatgaccgtcgtttcaacgaccccgcctggagtcagaacccactctacaaacgttatctacaaacctacctggcgtggcgcaaggaactccacgactggatcggcaacagcaaactgtccgaacaggacatcaatcgcgctcacttcgtgatcaccctgatgaccgaagccatggccccgaccaacagtgcggccaatccggcggcggtcaaacgcttcttcgaaaccggcggtaaaagcctgctcgacggcctcacacatctggccaaggacctggtaaacaacggcggcatgccgagccaggtggacatgggcgctttcgaagtcggcaagagtctggggacgactgaaggtgcagtggttttccgcaacgacgtcctcgaattgatccagtaccggccgaccaccgaacaggtgcatgagcgaccgctgctggtggtcccaccgcagatcaacaagttttatgtgtttgacctgagcccggataaaagcctggcgcgcttctgcctgagcaacaaccagcaaacctttatcgtcagctggcgcaacccgaccaaggcccagcgtgagtggggtctgtcgacttacatcgatgcgctcaaagaagccgtcgacgtagtttccgccatcaccggcagcaaagacatcaacatgctcggcgcctgctccggtggcattacctgcaccgcgctgctgggtcactacgccgctctcggcgagaagaaggtcaatgccctgacccttttggtcaccgtgctcgacaccaccctcgactcccaggttgcactgttcgtcgatgagaaaaccctggaagctgccaagcgtcactcgtatcaggccggcgtgctggaaggccgcgacatggccaaagtcttcgcctggatgcgccctaacgacctgatctggaactactgggtcaacaactacctgctgggtaacgagccaccggtcttcgacattcttttctggaacaacgacaccacccggttgcctgctgcgttccacggcgatctgatcgaaatgttcaaaaataacccactggtgcgcgccaatgcactcgaagtgagcggcacgccgatcgacctcaaacaggtcactgccgacatctactccctggccggcaccaacgatcacatcacgccctggaagtcttgctacaagtcggcgcaactgttcggtggcaaggtcgaattcgtgctgtccagccgcgggcatatccgcagcattctgaacccgccgggcaatccgaaatcacgttacatgaccagcaccgacatgccagccaccgccaacgagtggcaagaaaactcaaccaagcacaccgactcctggtggctgcactggcaggcctggcaggccgagcgctcgggcaaactgaaaaagtccccgaccagcctgggcaacaaggcctatccgtcaggagaagccgcgccgggcacgtatgtgcatgaacgttaa

SEQ ID NO: 12 - DNA, 842 bp, Ralstonia eutropha
cccgggcaagtaccttgccgacatctatgcgctggcgcgcacgcgcctggcgcgcgccggctgtaccgaggtctacggcggcgacgcctgcaccgtggccgacgccggtcgcttctactcctatcggcgcgatggcgtgaccggccgcatggccagcctggtctggctggcggactgagcccgccgctgcctcactcgtccttgcccctggccgcctgcgcgcgctcggcttcagccttgcgtcggcggcggccgggcgtgcccatgatgtagagcaccagcgccaccggcgccatgccatacatcaggaaggtggcaacgcctgccaccacgttgtgctcggtgatcgccatcatcagcgccacgtagagccagccaatggccacgatgtacatcaaaaattcatccttctcgcctatgctctggggcctcggcagatgcgagcgctgcataccgtccggtaggtcgggaagcgtgcagtgccgaggcggattcccgcattgacagcgcgtgcgttgcaaggcaacaatggactcaaatgtctcggaatcgctgacgattcccaggtttctccggcaagcatagcgcatggcgtctccatgcgagaatgtcgcgcttgccggataaaaggggagccgctatcggaatggacgcaagccacggccgcagcaggtgcggtcgagggcttccagccagttccagggcagatgtgccggcagaccctcccgctttgggggaggcgcaagccgggtccattcggatagcatctccccatgcaaagtgccggccagggcaatgcccggagccggttcgaatagtgacggcagagagacaatcaaatc

SEQ ID NO: 13 - DNA, 1680 bp, Pseudomonas sp.61-3
atgagtaacaagaatagcgatgacttgaatcgtcaagcctcggaaaacaccttggggcttaaccctgtcatcggcctgcgtggaaaagatctgctgacttctgcccgaatggttttaacccaagccatcaaacaacccattcacagcgtcaagcacgtcgcgcattttggcatcgagctgaagaacgtgatgtttggcaaatcgaagctgcaaccggaaagcgatgaccgtcgtttcaacgaccccgcctggagtcagaacccactctacaaacgttatctacaaacctacctggcgtggcgcaaggaactccacgactggatcggcaacagcaaactgtccgaacaggacatcaatcgcgctcacttcgtgatcaccctgatgaccgaagccatggccccgaccaacagtgcggccaatccggcggcggtcaaacgcttcttcgaaaccggcggtaaaagcctgctcgacggcctcacacatctggccaaggacctggtaaacaacggcggcatgccgagccaggtggacatgggcgctttcgaagtcggcaagagtctggggacgactgaaggtgcagtggttttccgcaacgacgtcctcgaattgatccagtaccggccgaccaccgaacaggtgcatgagcgaccgctgctggtggtcccaccgcagatcaacaagttttatgtgtttgacctgagcccggataaaagcctggcgcgcttctgcctgagcaacaaccagcaaacctttatcgtcagctggcgcaacccgaccaaggcccagcgtgagtggggtctgtcgacttacatcgatgcgctcaaagaagccgtcgacgtagtttccgccatcaccggcagcaaagacatcaacatgctcggcgcctgctccggtggcattacctgcaccgcgctgctgggtcactacgccgctctcggcgagaagaaggtcaatgccctgacccttttggtcagcgtgctcgacaccaccctcgactcccaggttgcactgttcgtcgatgagaaaaccctggaagctgccaagcgtcactcgtatcaggccggcgtgctggaaggccgcgacatggccaaagtcttcgcctggatgcgccctaacgacctgatctggaactactgggtcaacaactacctgctgggtaacgagccaccggtcttcgacattcttttctggaacaacgacaccacccggttgcctgctgcgttccacggcgatctgatcgaaatgttcaaaaataacccactggtgcgcgccaatgcactcgaagtgagcggcacgccgatcgacctcaaacaggtcactgccgacatctactccctggccggcaccaacgatcacatcacgccctggaagtcttgctacaagtcggcgcaactgttcggtggcaaggtcgaattcgtgctgtccagcagtgggcatatccagagcattctgaacccgccgggcaatccgaaatcacgttacatgaccagcaccgacatgccagccaccgccaacgagtggcaagaaaactcaaccaagcacaccgactcctggtggctgcactggcaggcctggcaggccgagcgctcgggcaaactgaaaaagtccccgaccagcctgggcaacaaggcctatccgtcaggagaagccgcgccgggcacgtatgtgcatgaacgttaa

SEQ ID NO: 14 - DNA, 32 bp, Artificial Sequence
  primer
gcgcgccaattgcccgggcaagtaccttgccg

SEQ ID NO: 15 - DNA, 21 bp, Artificial Sequence
  primer
catgatttgattgtctctctg

SEQ ID NO: 16 - DNA, 47 bp, Artificial Sequence
  primer
ggcagagagacaatcaaatcatgagtaacaagaatagcgatgacttg

SEQ ID NO: 17 - DNA, 75 bp, Artificial Sequence
  primer
gcgcgcactagtcggctgccgactggttgaaccaggccggcaggtcaggcttaacgttcatgcacatacgtgccc

SEQ ID NO: 18 - DNA, 46 bp, Artificial Sequence
  primer
cgagggtggtgtcgagcacggtgaccaaaagggtcagggcattgac

SEQ ID NO: 19 - DNA, 46 bp, Artificial Sequence
  primer
gtcaatgccctgacccttttggtcaccgtgctcgacaccaccctcg

SEQ ID NO: 20 - DNA, 57 bp, Artificial Sequence
  primer
cccggcgggttcagaatgctgcggatatgcccgcggctggacagcacgaattcgacc

SEQ ID NO: 21 - DNA, 57 bp, Artificial Sequence
  primer
ggtcgaattcgtgctgtccagccgcgggcatatccgcagcattctgaacccgccggg

SEQ ID NO: 22 - DNA, 45 bp, Artificial Sequence
  primer
cgccggcactcatgcaagcgttaacgttcatgcacatacgtgccc

(19) United States
(12) Patent Application Publication
(10) Pub. No.: US 2018/0346943 A1
(43) Pub. Date: Dec. 6, 2018

(54) POLYHYDROXYALKANOIC ACID HAVING FUNCTIONAL GROUP AT TERMINAL CARBOXY GROUP AND METHOD FOR PRODUCING THE SAME

(71) Applicant: KANEKA CORPORATION, Osaka-shi (JP)

(72) Inventors: Shingo KOBAYASHI, Settsu-shi (JP); Tetsuya FUJIKI, Settsu-shi (JP)

(73) Assignee: KANEKA CORPORATION, Osaka-shi (JP)

(21) Appl. No.: 16/100,688

(22) Filed: Aug. 10, 2018

Related U.S. Application Data
(63) Continuation of application No. PCT/JP2016/008842, filed on Dec. 22, 2016.

(30) Foreign Application Priority Data
Mar. 25, 2016 (JP) 2016-062278

(51) Int. Cl. C12P 7/62; C08G 63/06; C08G 63/91

(52) U.S. Cl. CPC C12P 7/625; C12N 1/20; C08G 63/912; C08G 63/06

(57) ABSTRACT

The present invention provides a novel polyhydroxyalkanoic acid having a functional group at a terminal carboxy group, which being capable of being chemically modified and easily controlled in reaction, and a method for production the same. The present polyhydroxyalkanoic acid includes a specific alkyne group, alkene group, thiol group, azide group or allyl group introduced at a terminal carboxy group. Further, the present production method comprises culturing a microorganism capable of producing a polyhydroxyalkanoic acid with use of an alcohol having an alkyne group, an alkene group, a thiol group, an azide group or an allyl group.

### TECHNICAL FIELD

The present invention relates to a novel polyhydroxyalkanoic acid having a functional group at a terminal carboxy group and a method for producing the same. Particularly, the present invention relates to a homopolymer of microorganism-producing R-3-hydroxyalkanoic acid having a hydroxy group at the 3-position or a copolymer [poly (R-3-hydroxyalkanoic acid)] and a method for producing the same.

### BACKGROUND ART

Polyhydroxyalkanoic acids (hereinafter abbreviated as “PHA”) are thermoplastic polyesters produced and accumulated as energy storage materials in cells of many microbial species. PHA produced from various natural carbon sources by microorganisms are completely biodegraded by microorganisms in the soil and water, so that the PHA is incorporated into the natural carbon cycle process. Therefore, it can be said that PHA is environmentally friendly plastics with little adverse effect on ecosystem. In recent years, as social problems with synthetic plastics become more serious from the viewpoint of environmental pollution, waste disposal, and petroleum resources, PHA has attracted attention as environmentally friendly green plastics, and it is desired to put them into practical use.

The PHA first discovered in microorganisms is polyhydroxybutyrate (hereinafter abbreviated as “PHB”) which is a homopolymer of 3-hydroxybutyric acid (hereinafter abbreviated as “3HB”). The PHB has high crystallinity, is hard and brittle due to its high degree of crystallinity, and rapidly thermally decomposes at a temperature (180° C.) near the melting point, so that it has problems that the melt processability is low and the practical range is extremely limited.

Therefore, in order to reduce the degree of crystallinity of the PHB and improve brittleness, attempts have been made to introduce another 3-hydroxyalkanoic acid into the PHB backbone. For example, it has been reported so far that a linear monomer having no side chain such as 3-hydroxypropionic acid (hereinafter abbreviated as “3HP”), 4-hydroxybutyric acid (hereinafter abbreviated as “4HB”) or 5-hydroxyvaleric acid (hereinafter abbreviated as “5HV”, or a monomer having a side chain such as lactic acid, 3-hydroxyvaleric acid (hereinafter abbreviated as “3HV”) or 3-hydroxyhexanoic acid (hereinafter abbreviated as “3HHx”) is introduced into the PHB backbone. The physical properties of PHA obtained greatly change depending on the types of monomers to be introduced and the copolymerization ratio of monomers. Basically, even if any monomer is introduced, the degree of crystallinity of PHB decreases, so that the melt processability is improved as compared with the case of PHB.

In order to further expand the use of PHA, it is necessary to develop a technology to produce a new PHA having physical properties significantly different from those of the PHA as described above. For that purpose, it is considered that it is effective not only to simply change the length of the PHA main chain or the size of the straight chain, but also to introduce some kind of functional groups into a side chain or terminal carboxy group of PHA.

So far, as an example of introducing a functional group into a side chain of PHA, Patent Document 1 reports that thioester is introduced into a side chain of medium-chain PHA having 6 to 14 carbon atoms. Further, Non-Patent Document 1 summarizes examples in which aromatic ring compounds such as a branched chain alkyl group, a cyclohexyl group, an alkyl halide, an acetoxy group, an ester, an alkoxy group, an epoxy group, a thiol group, a cyano group, a nitro group, a phenyl group, and a benzoyl group are introduced into a side chain of medium-chain PHA having 6 to 14 carbon atoms.

Introduction of these functional groups is important not only to greatly change the physical properties of PHA but also to provide a reaction starting point for further chemically modifying the above functional groups. For example, Non-Patent Document 3 reports that the double bond of PHA side chain is modified with a fluorescent substance or a peptide using thiol-ene click reaction. Also, Non-Patent Document 4 reports that a hydroxy group or carboxy group is introduced into the double bond of PHA side chain using thiol-ene click reaction, thereby changing the water repellency of the PHA. Further, Non-Patent Document 4 reports that the PHA can be crosslinked by reacting with a multi-branched structure compound. Furthermore, Non-Patent Document 5 reports an example in which a PHA having an azide group in its side chain is produced and the side chain is modified using alkyne-azide click reaction.

As described above, the method of introducing a functional group into a PHA and chemically modifying the PHA as a target is useful for changing the physical properties of the PHA. However, when the functional group of the PHA side chain is used as described above, it is very difficult to control the reaction. For example, in the case of crosslinking using the multi-branched structure compound, the PHA turns into a gel when the number of crosslinking points is large. However, when the number of crosslinking points is small, the effect of modifying physical properties by crosslinking cannot be obtained. Also, the fact that a plurality of reactive groups is present on one molecule of the PHA chain also had a problem that the product after the reaction becomes a mixture of various compounds.

On the other hand, Non-Patent Document 6 reports an example in which a functional group is introduced to a terminal carboxy group of PHA. In detail, Non-Patent Document 6 reports that when a microorganism of the genus Bacillus having a type IV PHA synthase gene is cultured in the presence of 1,3-propanediol, 2-propyn-1-ol, 3-mercapto-1-propanol, and benzyl alcohol, a 1-propanol group, a 1-propynyl group, a 1-propanethiol group, and a benzyl group are introduced at a terminal carboxy group of PHB, so that the molecular weight decreases as compared with the case of being cultured in the absence of these groups. Further, Non-Patent Document 6 reports that no decrease in molecular weight was observed even when the microorganism was cultured in the presence of 2-propen-1-ol or 3-butyn-1-ol.

### PRIOR ART DOCUMENT

### Patent Documents

Patent Document 1: WO2012/038572

### Non-Patent Documents

Non-Patent Document 1: Marta Tortajada, Luiziana Ferreira da Silva, Maria Auxiliadora Prieto, International Microbiology, vol. 16, pp. 1-15, 2013

Non-Patent Document 2: Henry E. Valentin, Pierre A. Berger, Kenneth J. Gruys, Maria Filomena de Andrade Rodrigues, Alexander Steinbuchel, Munhtien Tran, Jawed Asrar, Macromolecules, vol. 32, pp. 7389-7395, 1999

Non-Patent Document 3: Kenji Tajima, Kosuke Iwamoto, Yasuharu Sato, Ryosuke Sakai, Toshifumi Satoh, Tohru Dairi, Applied Microbiology and Biotechnology, vol. 100, pp. 4375-4383, 2016

Non-Patent Document 4: Alex C. Levine, Graham W. Heberlig, Christopher T. Nomura, International Journal of Biological Macromolecules, vol. 83, pp. 358-365, 2016

Non-Patent Document 5: Atahualpa Pinto, Jsessica H. Ciesla, Adriana Paulucci, Bradley P. Sutliff, Christopher T. Nomura, ACS Macro Letters, Vol. 5, pp. 215-219, 2016

Non-Patent Document 6: Manami Hyakutake, Satoshi Tomizawa, Imai Sugahara, Emi Murata, Kouhei Mizuno, Hideki Abe, Takeharu Tsuge, Polymer Degradation and Stability, vol. 117, pp. 90-96, 2015

### SUMMARY OF THE INVENTION

### Problems to be Solved by the Invention

As described above, Non-Patent Document 6 describes a compound in which a functional group is introduced at a terminal carboxy group of PHA, but only four compounds can be actually produced. For example, a propynyl group can be introduced for an alkynyl group, but a butynyl group cannot be introduced. An object of the present invention is to provide a method capable of producing various novel PHA each having a functional group at a terminal carboxy group, the PHA being capable of being chemically modified and easily controlled in reaction, and a PHA which has not been produced by a conventional method.

### Means for Solving the Problem

As a result of extensive studies to solve the above problems, the present inventors have succeeded in producing a PHA containing a specific functional group, not at a side chain of the PHA but at a terminal carboxy group of the PHA, and have completed the present invention.

The details of the present invention are as follows.

1. A polyhydroxyalkanoic acid that is a homopolymer or copolymer of R-3-hydroxyalkanoic acid having a repeating unit represented by a general formula (1) below:

[—C*HR¹—CH₂—CO—O—] (1)

(wherein R¹ is an alkyl group represented by CₙH₂ₙ₊₁, n is an integer of 1 to 15, and * represents an asymmetric carbon),

wherein

groups shown below are bonded to a terminal carboxy group depending on whether the polymer is a homopolymer or a copolymer:

(1) a group that bonds to the terminal carboxy group when the polymer is a homopolymer,

an alkynyl group having 4 to 8 carbon atoms,

an alkenyl group having 3 to 8 carbon atoms,

a mercaptoalkyl group having 2, 4 to 8 carbon atoms,

an azidated alkyl group having 3 to 8 carbon atoms, or

an allyl (poly)oxyalkyl group in which an alkyl group has 2 to 6 carbon atoms; and

(2) a group that bonds to the terminal carboxy group when the polymer is a copolymer,

an alkynyl group having 3 to 8 carbon atoms,

an alkenyl group having 3 to 8 carbon atoms,

a mercaptoalkyl group having 2 to 8 carbon atoms,

an azidated alkyl group having 3 to 8 carbon atoms, or

an allyl (poly)oxyalkyl group in which an alkyl group has 2 to 6 carbon atoms.

2. A polyhydroxyalkanoic acid that includes an alkyne group (which is synonym of alkynyl group), an alkene group (which is synonym of alkenyl group), a thiol group (which is synonym of mercapto group),or an azide group introduced at a terminal carboxy group and that is produced by microorganism,

wherein

the hydroxyalkanoic acid is composed of a plurality of kinds selected from 3-hydroxybutyric acid, 3-hydroxypropionic acid, 4-hydroxybutyric acid, 3-hydroxyvaleric acid, 5-hydroxyvaleric acid, 3-hydroxyhexanoic acid, 6-hydroxyhexanoic acid, 3-hydroxyheptanoic acid, 3-hydroxyoctanoic acid, 3-hydroxynonanoic acid, 3-hydroxydecanoic acid, 3-hydroxyundecanoic acid, and 3-hydroxydodecanoic acid.

3. A polyhydroxyalkanoic acid that includes an alkyne group, an alkene group, a thiol group or an azide group introduced at a terminal carboxy group and that is produced by microorganism,

wherein

the hydroxyalkanoic acid is composed of a single kind selected from 3-hydroxybutyric acid, 3-hydroxypropionic acid, 4-hydroxybutyric acid, 3-hydroxyvaleric acid, 5-hydroxyvaleric acid, 3-hydroxyhexanoic acid, 6-hydroxyhexanoic acid, 3-hydroxyheptanoic acid, 3-hydroxyoctanoic acid, 3-hydroxynonanoic acid, 3-hydroxydecanoic acid, 3-hydroxyundecanoic acid, and 3-hydroxydodecanoic acid;

the alkyne group is a butynyl group, a pentynyl group or a hexynyl group;

the alkene group is a propenyl group, a butenyl group, a pentenyl group or a hexenyl group; and

the thiol group is a mercaptoethyl group, a mercaptobutyl group, a mercaptopentyl group or a mercaptohexyl group.

4. The polyhydroxyalkanoic acid according to any one of above 1 to 3, which contains at least 3-hydroxybutyric acid as a monomer unit.

5. The polyhydroxyalkanoic acid according to above 4, which contains further 3-hydroxyhexanoic acid as a monomer unit.

6. A method for producing the polyhydroxyalkanoic acid according to any one of above 1 to 5,

the method comprising culturing a microorganism capable of producing a polyhydroxyalkanoic acid including an alkyne group, an alkene group, a thiol group, an azide group or an allyl group introduced at a terminal carboxy group with use of an alcohol having an alkyne group, an alkene group, a thiol group, an azide group or an allyl group.

7. A method for producing a polyhydroxyalkanoic acid including an alkyne group, an alkene group, a thiol group, an azide group or an allyl group introduced at a terminal carboxy group, the method comprising culturing a microorganism belonging to the genus Cupriavidus capable of producing a polyhydroxyalkanoic acid with use of an alcohol having 2 to 8 carbon atoms and having an alkyne group, an alkene group, a thiol group, an azide group or an allyl group.

8. The production method according to above 6 or 7, wherein the alcohol is a primary alcohol.

9. The production method according to any one of above 6 to 8, wherein the microorganism is a microorganism having a gene encoding a polyhydroxyalkanoate synthase derived from the genus Aeromonas, Ralstonia or Pseudomonas.

10. The production method according to above 8 or 9, wherein the microorganism is a microorganism belonging to the genus Cupriavidus.

11. The production method according to above 7 or 10, wherein the microorganism is a transformant including Cupriavidus necator as a host.

12. A compound wherein the alkyne group, the alkene group, the thiol group, the azide group or the allyl group at a terminus of the polyhydroxyalkanoic acid according to any one of above 1 to 5 is further chemically modified.

13. A molded article comprising the polyhydroxyalkanoic acid according to any one of above 1 to 5, or the compound according to 12 above.

### Effects of the Invention

According to the present invention, it is possible to produce a completely novel PHA having a specific functional group at a terminal carboxy group. The PHA of the present invention is used as an additive for producing a molded article such as a film or sheet so that the physical properties of a conventional PHA can be expected to be improved. In addition, it is considered that the above functional group is further chemically modified, thereby inducing the PHA into various structures.

### BRIEF DESCRIPTION OF THE DRAWINGS

FIG. 1 is ¹H-NMR chart of Example 8.

FIG. 2 is ¹H-NMR chart of Example 16.

FIG. 3 is ¹H-NMR chart of Comparative Example 1.

### MODE FOR CARRYING OUT THE INVENTION

Hereinafter, the present invention will be described in more detail.

The PHA of the present invention is a homopolymer or copolymer of R-3-hydroxyalkanoic acid (hereinafter abbreviated as “3HA”) (hereinafter each polymer is abbreviated as “P3HA”) having a repeating unit represented by the following general formula (1):

[—C*HR¹—CH₂—CO—O—] (1)

(wherein R¹ is an alkyl group represented by CₙH₂ₙ₊₁, n is an integer of 1 to 15, and * represents an asymmetric carbon), in which groups shown below are bonded to the terminal carboxy group depending on whether the P3HA is a homopolymer or a copolymer. Preferably, the following groups are bonded to the ester bond including the carboxy group, and the terminus of the PHA has a triple bond, a double bond, a mercapto group (thiol group), and an allyl group.

(1) A group that bonds to the terminal carboxy group when the polymer is a homopolymer

an alkynyl group having 4 to 8 carbon atoms, an alkenyl group having 3 to 8 carbon atoms, a mercaptoalkyl group having 2, 4 to 8 carbon atoms, an azidated alkyl group having 3 to 8 carbon atoms, or an allyl (poly)oxyalkyl group in which an alkyl group has 2 to 6 carbon atoms.

(2) A group that bonds to the terminal carboxy group when the polymer is a copolymer

an alkynyl group having 3 to 8 carbon atoms, an alkenyl group having 3 to 8 carbon atoms, a mercaptoalkyl group having 2 to 8 carbon atoms, an azidated alkyl group having 3 to 8 carbon atoms, or an allyl (poly)oxyalkyl group in which an alkyl group has 2 to 6 carbon atoms.

Non-Patent Document 6 described above merely discloses a homopolymer of 3HB in which n=1 in the above general formula (1) and which has a propynyl group (an alkynyl group having 3 carbon atoms) and a mercaptopropyl group (a thiol group having an alkyl group having 3 carbon atoms) at a terminal carboxy group. The type of the functional group in Non-Patent Document 6 is different from that in the present invention. Further, in both cases, the microbial species to be used are different from each other.

That is, the PHA of the present invention is a polyester resin produced from a microorganism, the PHA containing 3HA as a main monomer unit and being referred to as “P3HA”. Examples of the monomer unit constituting the PHA of the present invention include such as 3HB (n=1 of the alkyl group R¹ represented by CₙH₂ₙ₊₁ in the above general formula (1)), 3HV (n=2), 3HHx (n=3), 3-hydroxyheptanoic acid (n=4), 3-hydroxyoctanoic acid (n=5), 3-hydroxynonanoic acid (n=6), 3-hydroxydecanoic acid (n=7), 3-hydroxy undecanoic acid (n=8), and 3-hydroxydodecanoic acid (n=9).

The monomer unit in the PHA of the present invention may be one kind or may be plural kinds. In the case of containing plural kinds of monomer units, two or more kinds of 3HAs may be copolymerized, or 4-hydroxyalkanoic acid such as 4HB may be copolymerized with one or two or more kinds of 3HAs. The PHA of the present invention preferably contains at least 3HB as a monomer unit. The PHA of the present invention may be a PHB consisting only of the above 3HB as a monomer unit or may be a copolymer composed of 3HB and other monomer units. In addition to the above-mentioned monomer units, examples of the monomer units other than 3HB include 3HP, 4HB, 5HV, and 6-hydroxyhexanonate (hereinafter abbreviated as “6HHx”). Examples of the copolymers include poly(3HB-co-3HHx) further having 3HHx (hereinafter abbreviated as “PHBH”), poly(3HB-co-3HV) further having 3HV, and poly(3HB-co-4HV) further having 4HV. Of these, the PHBH is preferred.

When the PHA of the present invention is a copolymer, the copolymerization ratio of each monomer unit is not particularly limited. When 3HB is contained as the monomer unit, the copolymerization ratio thereof is more preferably 50 mol % or more, still more preferably 60 mol % or more, yet still more preferably 70 mol % or more, and particularly preferably 80 mol % or more. Further, when the PHA of the present invention is PHBH, the lower limit of the copolymerization ratio of 3HHx is preferably 1 mol %, more preferably 2 mol %, and still more preferably 3 mol %. The upper limit thereof is preferably 20 mol %, more preferably 15 mol %, and still more preferably 12 mol %.

As described above, the PHA of the present invention is characterized in that the PHA includes, as a specific functional group, an alkynyl group, an alkenyl group, a thiol group (mercapto group), an azide group or an allyl group introduced at the terminal carboxy group of the polymer main chain. The terminal carboxy group of the PHA of the present invention has an alkynyl group, an alkenyl group, a thiol group, and an azide group via an alkyl chain, and also has an allyl group via an oxyalkyl chain.

Examples of the alkynyl group having 3 to 8 carbon atoms include a straight-chain or branched propynyl group, a straight-chain or branched butynyl group, a straight-chain or branched pentynyl group, a straight-chain or branched hexynyl group, a straight-chain or branched heptynyl group, and a straight-chain or branched octynyl group. The non-branched chain, i.e., the straight-chain is preferred. The number of carbon atoms is preferably from 3 to 6. Of these, preferable examples include a propynyl group, a butynyl group, and a hexynyl group.

Examples of the alkenyl group having 3 to 8 carbon atoms include a straight-chain or branched propenyl group, a straight-chain or branched butenyl group, a straight-chain or branched pentenyl group, a straight-chain or branched hexenyl group, a straight-chain or branched heptenyl group, and a straight-chain or branched octenyl group. The non-branched chain, i.e., the straight-chain is preferred. The number of carbon atoms is preferably from 3 to 6. Of these, preferable examples include a propenyl group, a butenyl group, and a hexenyl group.

Examples of the mercaptoalkyl group having 2 to 8 carbon atoms include a straight-chain or branched mercaptoethyl group, a straight-chain or branched mercaptopropyl group, a straight-chain or branched mercaptobutyl group, a straight-chain or branched mercaptopentyl group, a straight-chain or branched mercaptohexyl group, a straight-chain or branched mercaptoheptyl group, and a straight-chain or branched mercaptooctyl group. The non-branched chain, i.e., the straight-chain is preferred. The number of carbon atoms is preferably from 2 to 6, and more preferably from 3 to 6. Preferable examples thereof include a mercaptoethyl group and a mercaptopropyl group.

Examples of the azidated alkyl group having 3 to 8 carbon atoms include a straight-chain or branched azidated propyl group, a straight-chain or branched azidated butyl group, a straight-chain or branched azidated pentyl group, a straight-chain or branched azidated hexyl group, and a straight-chain or branched azidated heptyl group. The non-branched chain, i.e., the straight-chain is preferred. The number of carbon atoms is preferably from 3 to 6. Of these, preferable examples include an azidated propyl group and an azidated butyl group.

Examples of the allyl (poly)oxyalkyl group in which the alkyl group has 2 to 6 carbon atoms include allyl (poly)oxyalkyl groups having 1 to 3 oxyalkyl groups, preferably having one oxyalkyl group. Examples of the oxyalkyl group include an oxyethyl group, an oxypropyl group, an oxybutyl group, an oxypentyl group, and an oxyhexyl group. Of these, preferred examples include an oxyethyl group, an oxypropyl group, and an oxybutyl group. The non-branched chain, i.e., the straight-chain is preferred. The total carbon number of the allyl (poly)oxyalkyl group is preferably from 2 to 6, and examples of such allyl (poly)oxyalkyl groups include an allyloxyethyl group, an allyloxypropyl group, and an allyloxybutyl group.

As described above, the PHA of the present invention characteristically includes a specific functional group at a terminal carboxy group, and the polymer side chain may further have a functional group. However, when the PHA of the present invention is further chemically modified to synthesize a new derivative, it is preferable that a functional group is present only at a terminal carboxy group from the viewpoint of reaction control.

Although the molecular weight of the PHA of the present invention is not limited, a PHA having a relatively low molecular weight tends to be obtained when the production method of the present invention described below is used. For example, the weight average molecular weight (M_{w}) may be about from 5000 to 20000000. Depending on the intended use, it may be about from 8000 to 300000. Further, it may be allowed to be about from 10000 to 100000. Further, the number average molecular weight (Mₙ) may be about from 3000 to 1500000. Depending on the intended use, it may be about from 5000 to 1000000. Further, it may be allowed to be about from 7000 to 800000.

The above-described method for producing the PHA of the present invention is, for example, a method of culturing a microorganism capable of synthesizing a PHA (hereinafter referred to as “microorganism of the present invention”) using an alcohol having the alkynyl group, the alkenyl group, the thiol group, the azide group or the allyl group, in order to introduce the specific functional group at a terminal carboxy group (hereinafter referred to as “production method of the present invention”). The alcohol is preferably a primary alcohol.

Examples of the alcohol include, for example, 2-propyn-1-ol, 3-butyn-1-ol, 4-pentyn-1-ol, 5-hexyn-1-ol; 2-propen-1-ol, 3-buten-1-ol, 4-penten-1-ol, 5-hexen-1-ol; 2-mercaptoethanol, 3-mercaptopropanol, 4-mercaptobutanol, 5-mercaptopentanol, 6-mercaptohexanol; 4-azidobutan-1-ol, 5-azidopentan-1-ol, 6-azidohexan-1-ol; ethylene glycol monoallyl ether, propylene glycol monoallyl ether, tetramethylene glycol monoallyl ether, and pentamethylene glycol monoallyl ether. Of these, preferred examples include such as 2-propen-1-ol, 3-buten-1-ol, 5-hexen-1-ol, 2-propyn-1-ol, 3-butyn-1-ol, 5-hexyn-1-ol, 2-mercaptoethanol, 3-mercaptopropanol, and ethylene glycol monoallyl ether. In addition to this, an alcohol having a branched structure in its alkyl chain moiety, an alcohol having a plurality of hydroxy groups, or an alcohol having a plurality of alkynyl groups, alkenyl groups, thiol groups, azide groups, or allyl groups may be used. These alcohols may be used singly or in combination of two or more kinds thereof.

In the production method of the present invention, it is considered that the alcohol functions as a terminator in the chain transfer reaction to synthesize PHA in bacterial cells of microorganisms. In this case, the kind of alcohol functioning as a terminator depends on the substrate specificity of a PHA synthase possessed by the microorganism of the present invention to alcohol. In the production method of the present invention, the microorganism of the present invention is preferably a microorganism having a gene encoding a PHA synthase derived from the genera Aeromonas, Ralstonia or Pseudomonas (hereinafter abbreviated as “PHA synthase gene”). More specifically, more preferable examples of the PHA synthase gene include, but are not limited to, a PHA synthase gene which consists of the amino acid sequence represented by SEQ ID NO: 1 and is derived from Aeromonas caviae and in which asparagine at position 149 is artificially replaced with serine and aspartic acid at position 171 is artificially replaced with glycine; a PHA synthase gene which consists of the amino acid sequence represented by SEQ ID NO: 2 and is derived from Ralstonia eutropha; and a PHA synthase gene which consists of the amino acid sequence represented by SEQ ID NO: 3 and is derived from Pseudomonas Sp. 61-3 and in which serine at position 325 is artificially replaced with threonine, serine at position 477 is artificially replaced with arginine, and glutamine at position 481 is artificially replaced with arginine. A PHA synthase derived from the genus Aeromonas is known to have a hydroxyalkanoic acid CoA having 3 to 6 carbon atoms as a substrate. Accordingly, by using a microorganism having the PHA synthase gene derived from the genus Aeromonas, it is possible to produce a copolymerized PHA consisting of homopolymers of 3HB, 3HP, 4HB, 3HV, 5HV, and 3HHx, or monomer units of these homopolymers. Further, a PHA synthase derived from the genus Ralstonia is known to have a hydroxyalkanoic acid CoA having 3 to 5 carbon atoms as a substrate. Accordingly, by using a microorganism having the PHA synthase gene derived from the genus Ralstonia, it is possible to produce a copolymerized PHA consisting of homopolymers of 3HB, 3HP, 4HB, 3HV, and 5HV, or monomer units of these homopolymers. Further, a PHA synthase derived from the genus Pseudomonas has a hydroxyalkanoic acid CoA having 3 to 12 carbon atoms as a substrate. Accordingly, by using a microorganism having the PHA synthase gene derived from the genus Pseudomonas, it is possible to produce a copolymerized PHA consisting of homopolymers of 3HB, 3HP, 4HB, 3HV, 5HV, 3HHx, 6HHx, 3-hydroxyheptanoic acid, 3-hydroxyoctanoic acid, 3-hydroxynonanoic acid, 3-hydroxydecanoic acid, 3-hydroxyundecanoic acid, and 3-hydroxydodecanoic acid, or monomer units of these homopolymers.

The microbial species in the present invention are not particularly limited, and may be either bacteria or fungi. Examples of the microbial species include microorganisms belonging to the genera Acinetobacter, Aeromonas, Alcaligenes, Allochromatium, Azorhizobium, Azotobacter, Bacillus, Burkholderia, Candida, Caulobacter, Chromobacterium, Comamonas, Cupriavidus, Ectothiorhodospira, Escherichia, Klebsiella, Methylobacterium, Nocardia, Paracoccus, Pseudomonas, Ralstonia, Rhizobium, Rhodobacter, Rhodococcus, Rhodospirillum, Rickettsia, Saccharomyces, Sinorhizobium, Sphingomonas, Synechocystis, Thiococcus, Thiocystis, Vibrio, Wautersia, and Zoog/Loea. Of these microorganisms, microorganisms belonging to the genera Aeromonas, Alcaligenes, Cupriavidus, Escherichia, Pseudomonas, Ralstonia, and the like are preferred, microorganisms belonging to the genera Cupriavidus, Escherichia, Ralstonia are more preferred, and microorganisms belonging to the genus Cupriavidus are still more preferred. As the microorganism of the present invention, Cupriavidus necator is particularly preferred.

When the microorganism of the present invention does not have the PHA synthase gene originally or when the PHA synthase gene originally possessed by the microorganism is not a desired PHA synthase gene, for example, it is also possible to use a transformant in which the preferred PHA synthase gene described above is introduced into a microorganism as a host by genetic recombination method. As the method of introducing the PHA synthase gene into the host, the gene may be retained by plasmids or may be introduced into an arbitrary position of a chromosome. In this case, it is preferable that the PHA synthase gene originally possessed by the host loses its function. Example of the method of deleting the function of the PHA synthase gene include a method of deleting the PHA synthase gene in full length or partial length and a method of deleting the function of the PHA synthase produced by the addition, deletion or substitution of a base to the PHA synthase gene. Regarding specific methods for DNA insertion or substitution, for example, the description in Green, M. R. and Sambrook, J., 2012, Molecular Cloning: A Laboratory Manual fourth Ed., Cold Spring Harbor Laboratory Press, Cold Spring Harbor, N.Y. may be used as a reference.

In the production method of the present invention, the above-mentioned alcohol may be used singly as a carbon source for producing PHA; however, when the alcohol is used for culturing in large quantities, there is a high possibility that the growth of the microorganism is affected. Thus, it is preferable to use the alcohol in combination with carbon sources other than the alcohol. As the carbon sources other than the alcohol, any raw materials can be used as long as they are carbon sources that can be utilized by the microorganism of the present invention. Such carbon sources are not particularly limited, and preferred are sugars such as glucose, fructose, and sucrose; fats and oils such as palm oil, palm kernel oil (hereinafter abbreviated as “PKO”), corn oil, coconut oil, olive oil, soybean oil, rapeseed oil, and jatropha oil; fractionated oils thereof or purified byproducts thereof; fatty acids such as lauric acid, oleic acid, stearic acid, palmitic acid, myristic acid; and derivatives thereof. Further, a yeast extract, polypeptone, and the like may also be used. More preferred are vegetable fats and oils such as palm oil and palm kernel oil, or palm olein, palm double olein or palm kernel olein, which is a low-melting-point fraction obtained by fractionating palm oil or palm kernel oil; and a purified byproduct of an oil and fat, such as a PFAD (palm fatty acid distillate), a PKFAD (palm kernel fatty acid distillate) or a fatty acid distilled product of rapeseed oil. Especially preferred is the purified byproduct of the oil and fat from the viewpoint of avoiding competition with food.

In the production method of the present invention, it is preferable to culture the microorganism using a medium containing the alcohol, a carbon source other than alcohol, a nitrogen source which is a nutrient source other than the carbon source, inorganic salts, and other organic nutrition sources. Examples of the nitrogen source include ammonia, ammonium chloride, urea, ammonium salts such as ammonium sulfate and ammonium phosphate, peptone, a meat extract, and a yeast extract. Examples of the inorganic salts include potassium dihydrogenphosphate, disodium hydrogenphosphate, magnesium phosphate, magnesium sulfate, and sodium chloride. Examples of the other organic nutrient sources include amino acids such as glycine, alanine, serine, threonine and proline, and vitamins such as vitamins B1, B12, and C.

In the production method of the present invention, the concentration of the alcohol in the medium is not particularly limited. In order to efficiently produce the PHA of the present invention, the lower limit of the concentration is preferably 0.01 g/L, more preferably 0.05 g/L, and still more preferably 0.1 g/L. The upper limit of the concentration is preferably about 5 g/L, more preferably 3 g/L, and still more preferably 2 g/L, from the viewpoint of suppressing the influence on the growth of the microorganism as described above. When mercapto alcohol is used as the alcohol, it is particularly preferable to use the alcohol at a concentration of 0.8 g/L or less.

Other culture conditions such as culture temperature, culture time, pH during culture, and medium may be the culture conditions usually used in the microorganism of the present invention.

In the production method of the present invention, the method of recovering a PHA from bacterial cells is not particularly limited, and can be performed by the following method, for example. After completion of the culture, the bacterial cells are separated from the culture solution by a centrifugal separator or the like, the bacterial cells are washed with distilled water, methanol and the like and dried. From the dried bacterial cells, a PHA is extracted using an organic solvent such as chloroform. From the organic solvent solution containing the PHA, bacterial cell components are removed by filtration or the like. A poor solvent such as methanol or hexane is added to the filtrate to precipitate the PHA. Further, the supernatant fluid is removed by filtration or centrifugation and the recovered fluid is dried, thereby recovering the PHA.

The PHA of the present invention can be further rearranged to an arbitrary compound by a chemical reaction, i.e., can also be chemically modified, via a specific alkenyl group, an alkynyl group, a thiol group, an azide group or an allyl group which has been introduced at the terminal carboxy group. For example, when the PHA of the present invention has an alkenyl group or an alkynyl group at the terminal carboxy group, a compound containing a thiol group can be rearranged by thiol-ene click reaction or thiol-in click reaction. Conversely, when the PHA of the present invention has a thiol group at the terminal carboxy group, a compound containing an alkenyl group or an alkynyl group can be rearranged by thiol-ene click reaction or thiol-in click reaction. Alternatively, when the PHA of the present invention has an alkynyl group at the terminal carboxy group, a compound containing an azide group can be rearranged by an alkyne-azide click reaction. Conversely, when the PHA of the present invention has an azide group at the terminal carboxy group, a compound containing an alkynyl group can be rearranged by an alkyne-azide click reaction. Derivatives obtained by such chemical modification are also within the scope of the present invention. Further, a resin-molded article containing the PHA of the present invention and the compounds as the derivatives of the PHA is also within the scope of the present invention. In the resin-molded article, the PHA of the present invention or the compounds as the derivatives of the PHA may be used singly, or may be mixed with a conventionally known PHA for use.

When the PHA of the present invention has a functional group only at the terminus of the polymer main chain, it is possible to limit the product after the reaction, and thus this is more preferred. For example, a PHA having a double bond in a side chain is caused to react with a PHA having a thiol group at the terminal carboxy group, thereby inducing into a graft polymer. Further, a PHA having an alkene group at the terminal carboxy group is rearranged to a thiol compound having a multi-branched structure such as pentaerythritol tetrakis (3-mercaptopropionate), thereby inducing into a multi-branched PHA. Alternatively, it is possible to modify the surface of the resin molded product by processing a resin composition containing a PHA having an alkene group introduced at the terminal carboxy group into a film, a sheet, a non-woven fabric or the like, and rearranging the thiol compound.

Further, a dendrimer is constituted using the PHA of the present invention so that it is possible to provide a medical material having high biocompatibility and an agricultural material having an appropriate sustained release property.

This application claims the benefit of priority to Japanese Patent Application No. 2016-062278, filed on Mar. 25, 2016. The entire contents of the specifications of Japanese Patent Application No. 2016-062278, filed on Mar. 25, 2016 are incorporated herein by reference.

### EXAMPLES

Hereinafter, the present invention will be described more specifically with reference to examples, but the present invention is not limited to these examples. Note that, the breeding of strains, the method of analyzing the copolymerization ratio of the monomer units contained in PHA, and the method of analyzing the molecular weight of PHA are described below.

### Breeding of Strains

The gene manipulation in examples of the present specification can be performed by the method described in Green, M. R. and Sambrook, J. (2012) as aforementioned. The enzyme, cloning host and the like used in the gene manipulation can be purchased from the market supplier, and can be used in accordance with the instructions thereof. The enzymes used in examples and the like are not particularly limited as long as they can be used for gene manipulation.

### Method of Analyzing Copolymerization Ratio of Monomer Units Contained in PHA

The copolymerization ratio of the monomer units contained in PHA was analyzed using NMR. Specifically, 2 mg of the obtained PHA was dissolved in 2 mL of deuterated chloroform and the resultant mixture was transferred to a sample tube for measurement. From the area of each detected peak, the copolymerization ratio of the monomer units was calculated.

### Method of Analyzing Molecular Weight of PHA

The molecular weight of PHA was analyzed by the gel permeation chromatography method. A PHA was dissolved in chloroform at a concentration of 1.5 g/L and filtered through a filter (Φ=0.2 μm) to obtain a filtrate, which was used as an analysis sample. The used measurement system was GPC system of Shimadzu Corporation. Two Shodex GPC K-806L columns (SHOWA DENKO K.K.) were used in a state of being connected in series, and a column oven was set at 40° C. Chloroform was used as a mobile phase, and the flow rate was 1.0 mL/min. As molecular weight standards, polystyrenes each having a molecular weight of about 7,000,000, about 1,900,000, about 350,000, about 190,000, about 30,000, and about 2,000 polystyrenes were used. A calibration curve was prepared from the analytical results of 6 standards, and the molecular weight (a weight average molecular weight M_{w} and a number average molecular weight Mₙ) of the PHA was calculated based on the calibration curve.

### Confirmation of Structure of Terminal Carboxy Group of PHA

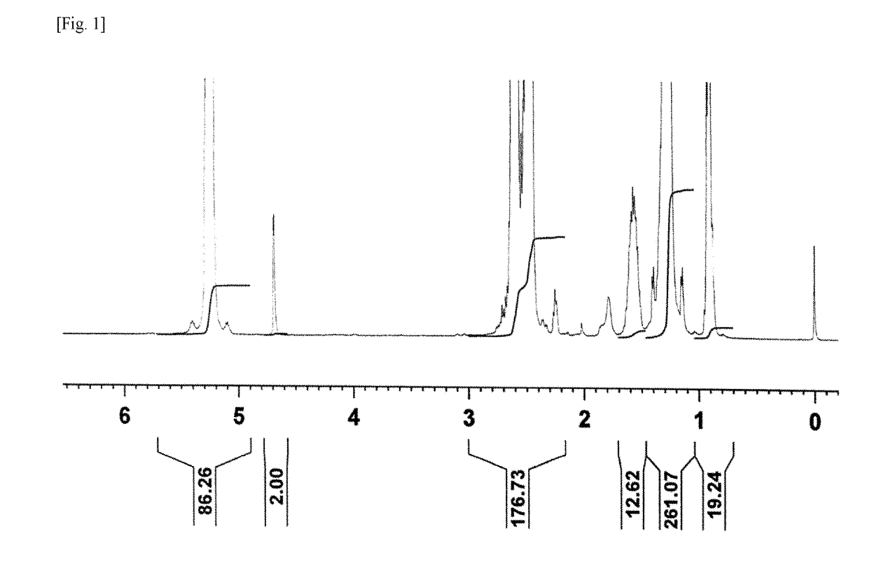

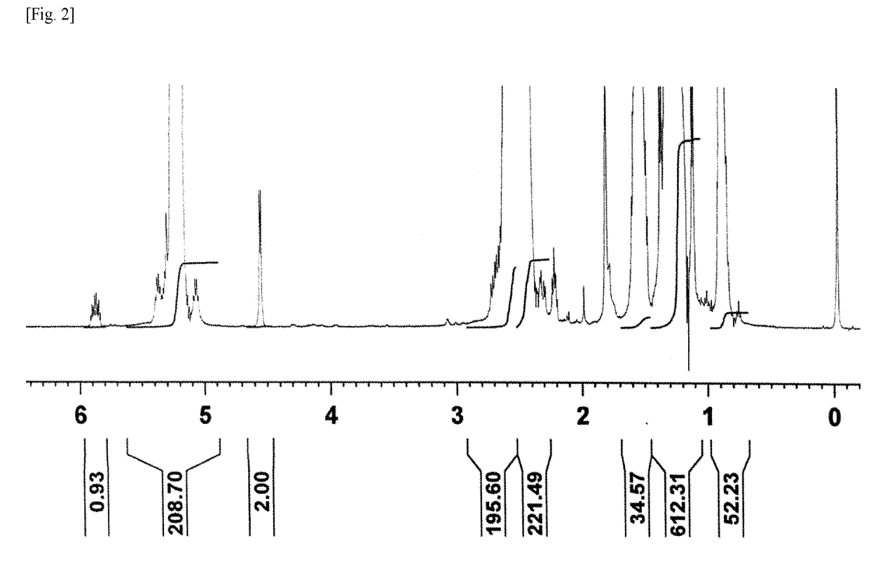

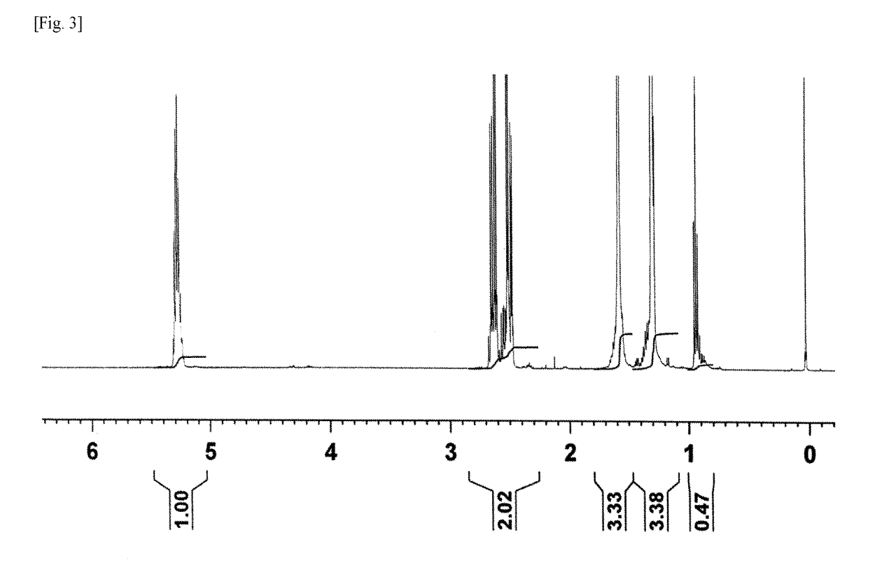

The ¹H-NMR was used to confirm whether a predetermined group was introduced at a terminal carboxy group. The measurement of ¹H-NMR was performed under the conditions of 500 MHz, room temperature, and the number of scanning of about 256 using an NMR instrument (manufactured by JEOL Ltd.). Data analysis was performed using ALICE 2 for windows ver. 4. Specific results of Examples 8 and 16 below are shown in FIGS. 1 and 2. For comparison, the result of Comparative Example 1 is also shown in FIG. 3.

### Example 1

Production of PHA by KNK-005 trc-phaJ4bΔphaZ1,2,6 Strain in Medium Containing 0.2 g/L of 2-Propen-1-ol

As the microorganism, KNK-005 trc-phaJ4b/ΔphaZ1,2,6 strain described in WO2015/115619 was used. The KNK-005 trc-phaJ4b/ΔphaZ1,2,6 strain is a strain in which the full-lengths of the phaZ1 and phaZ6 genes on the chromosome are deleted, the 16th codon to stop codon of the phaZ2 gene are deleted, a mutant PHA synthase gene derived from Aeromonas caviae represented by SEQ ID NO: 4 is present on the chromosome, and the expression regulatory sequence represented by SEQ ID NO: 5 is inserted into the upstream of the phaJ4b gene.

### Culture

The above microorganism was cultured under the following conditions.

The seed culture medium used contained 10 g/L of meat extract; 10 g/L of bacto tryptone; 2 g/L of yeast extract; 9 g/L of sodium dihydrogen phosphate dodecahydrate; and 1.5 g/L of dipotassium hydrogenphosphate.

The PHA production medium used contained 11 g/L of disodium hydrogen phosphate dodecahydrate; 1.9 g/L of dipotassium hydrogenphosphate; 1.3 g/L of ammonium sulfate; 5 mL/L of magnesium solution; and 1 mL/L of trace metal salt solution. The magnesium solution was prepared by dissolving 200 g/L of magnesium sulfate heptahydrate in water. The trace metal salt solution was prepared by dissolving 0.218 g/L of cobalt chloride hexahydrate, 16.2 g/L of iron (III) chloride hexahydrate, 10.3 g/L of calcium chloride dihydrate, 0.118 g/L of nickel chloride hexahydrate, and 0.156 g/L of copper sulfate pentahydrate in 0.1 N hydrochloric acid.

A glycerol stock solution (50 μL) of the strain was inoculated into 10 mL of the seed culture medium, followed by shaking culture at 30° C. for 24 hours. The obtained culture solution was used as a preculture solution.

The PHA production culture was performed in a flask. The PHA production medium (50 mL) was placed in a 500-mL shake flask. Just before the inoculation, 250 μL of the magnesium solution, 50 μL of the trace metal solution, and 1 g of PKO were added to the shake flask and further 2-propen-1-ol was added so as to be 0.2 g/L. After preparation of the medium, 500 μL of the preculture solution was inoculated into the shake flask, followed by shaking culture at 30° C. for 72 hours.

### Purification

After completion of the culture, the bacterial cells were recovered by centrifugation and suspended in water. Then, sodium lauryl sulfate was added to the suspension so as to have a final concentration of 3% (w/v). The prepared bacterial cell solution was treated with ultrasound while cooling with ice, thereby crushing the bacterial cells. The precipitate of PHA was collected from the crushed bacterial cell solution by centrifugation and washed once with water and ethanol. Then, the precipitate was vacuum dried at 60° C. for 2 hours to obtain a purified PHA.

Regarding the obtained PHA, the copolymerization ratio of the monomer units and the molecular weight were analyzed. The results are shown in Table 1.

### Example 2

Production of PHA by KNK-005 trc-phaJ4b/ΔphaZ1,2,6 Strain in Medium Containing 1.0 g/L of 2-Propen-1-ol

The KNK-005 trc-phaJ4b/ΔphaZ1,2,6 strain was cultured and the resultant was purified in the same manner as in Example 1 to obtain a purified PHA. However, the final concentration of 2-propen-1-ol added to the medium in the PHA production culture was 1.0 g/L. Regarding the obtained PHA, the copolymerization ratio of the monomer units and the molecular weight were analyzed. The results are shown in Table 1.

### Example 3

Production of PHA by KNK-005 trc-phaJ4b/ΔphaZ1,2,6 Strain in Medium Containing 0.2 g/L of 3-Buten-1-ol

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, 3-buten-1-ol was added to the medium so as to have a final concentration of 0.2 g/L. Regarding the obtained PHA, the copolymerization ratio of the monomer units and the molecular weight were analyzed. The results are shown in Table 1.

### Example 4

Production of PHA by KNK-005 trc-phaJ4b/ΔphaZ1,2,6 Strain in Medium Containing 1.0 g/L of 3-Buten-1-ol

The KNK-005 trc-phaJ4b/ΔphaZ1,2,6 strain was cultured and the resultant was purified in the same manner as in Example 3 to obtain a purified PHA. However, the final concentration of 3-buten-1-ol added to the medium in the PHA production culture was 1.0 g/L. Regarding the obtained PHA, the copolymerization ratio of the monomer units and the molecular weight were analyzed. The results are shown in Table 1.

### Example 5

Production of PHA by KNK-005 trc-phaJ4b/ΔphaZ1,2,6 Strain in Medium Containing 0.2 g/L of 5-Hexen-1-ol

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, 5-hexen-1-ol was added to the medium so as to have a final concentration of 0.2 g/L. Regarding the obtained PHA, the copolymerization ratio of the monomer units and the molecular weight were analyzed. The results are shown in Table 1.

### Example 6

Production of PHA by KNK-005 trc-phaJ4b/ΔphaZ1,2,6 Strain in Medium Containing 1.0 g/L of 5-Hexen-1-ol

The KNK-005 trc-phaJ4b/ΔphaZ1,2,6 strain was cultured and the resultant was purified in the same manner as in Example 5 to obtain a purified PHA. However, the final concentration of 5-hexen-1-ol added to the medium in the PHA production culture was 1.0 g/L. Regarding the obtained PHA, the copolymerization ratio of the monomer units and the molecular weight were analyzed. The results are shown in Table 1.

### Example 7

Production of PHA by KNK-005 trc-phaJ4b/ΔphaZ1,2,6 Strain in Medium Containing 0.2 g/L of 2-Propyn-1-ol

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, 2-propyn-1-ol was added to the medium so as to have a final concentration of 0.2 g/L. Regarding the obtained PHA, the copolymerization ratio of the monomer units and the molecular weight were analyzed. The results are shown in Table 1.

### Example 8

Production of PHA by KNK-005 trc-phaJ4b/ΔphaZ1,2,6 Strain in Medium Containing 1.0 g/L of 2-Propyn-1-ol

The KNK-005 trc-phaJ4b/ΔphaZ1,2,6 strain was cultured and the resultant was purified in the same manner as in Example 7 to obtain a purified PHA. However, the final concentration of 2-propyn-1-ol added to the medium in the PHA production culture was 1.0 g/L. Regarding the obtained PHA, the copolymerization ratio of the monomer units and the molecular weight were analyzed. The results are shown in Table 1.

### Example 9

Production of PHA by KNK-005 trc-phaJ4b/ΔphaZ1,2,6 Strain in Medium Containing 0.2 g/L of 3-Butyn-1-ol

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, 3-butyn-1-ol was added to the medium so as to have a final concentration of 0.2 g/L. Regarding the obtained PHA, the copolymerization ratio of the monomer units and the molecular weight were analyzed. The results are shown in Table 1.

### Example 10

Production of PHA by KNK-005 trc-phaJ4b/ΔphaZ1,2,6 Strain in Medium Containing 1.0 g/L of 3-Butyn-1-ol

The KNK-005 trc-phaJ4b/ΔphaZ1,2,6 strain was cultured and the resultant was purified in the same manner as in Example 9 to obtain a purified PHA. However, the final concentration of 3-butyn-1-ol added to the medium in the PHA production culture was 1.0 g/L. Regarding the obtained PHA, the copolymerization ratio of the monomer units and the molecular weight were analyzed. The results are shown in Table 1.

### Example 11

Production of PHA by KNK-005 trc-phaJ4b/ΔphaZ1,2,6 Strain in Medium Containing 0.2 g/L of 5-Hexyn-1-ol

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, 5-hexyn-1-ol was added to the medium so as to have a final concentration of 0.2 g/L. Regarding the obtained PHA, the copolymerization ratio of the monomer units and the molecular weight were analyzed. The results are shown in Table 1.

### Example 12

Production of PHA by KNK-005 trc-phaJ4b/ΔphaZ1,2,6 Strain in Medium Containing 1.0 g/L of 5-Hexyn-1-ol

The KNK-005 trc-phaJ4b/ΔphaZ1,2,6 strain was cultured and the resultant was purified in the same manner as in Example 11 to obtain a purified PHA. However, the final concentration of 5-hexyn-1-ol added to the medium in the PHA production culture was 1.0 g/L. Regarding the obtained PHA, the copolymerization ratio of the monomer units and the molecular weight were analyzed. The results are shown in Table 1.

### Example 13

Production of PHA by KNK-005 trc-phaJ4b/ΔphaZ1,2,6 Strain in Medium Containing 0.2 g/L of 2-Mercaptoethanol

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, 2-mercaptoethanol was added to the medium so as to have a final concentration of 0.2 g/L. Regarding the obtained PHA, the copolymerization ratio of the monomer units and the molecular weight were analyzed. The results are shown in Table 1.

### Example 14

Production of PHA by KNK-005 trc-phaJ4b/ΔphaZ1,2,6 Strain in Medium Containing 0.2 g/L of 3-Mercaptopropanol

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, 3-mercaptopropanol was added to the medium so as to have a final concentration of 0.2 g/L. Regarding the obtained PHA, the copolymerization ratio of the monomer units and the molecular weight were analyzed. The results are shown in Table 1.

### Example 15

Production of PHA by KNK-005 trc-phaJ4b/ΔphaZ1,2,6 Strain in Medium Containing 0.2 g/L of Ethylene glycol monoallyl ether

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, ethylene glycol monoallyl ether was added to the medium so as to have a final concentration of 0.2 g/L. Regarding the obtained PHA, the copolymerization ratio of the monomer units and the molecular weight were analyzed. The results are shown in Table 1.

### Example 16

Production of PHA by KNK-005 trc-phaJ4b/ΔphaZ1,2,6 Strain in Medium Containing 1.0 g/L of Ethylene glycol monoallyl ether

The KNK-005 trc-phaJ4b/ΔphaZ1,2,6 strain was cultured and the resultant was purified in the same manner as in Example 15 to obtain a purified PHA. However, the final concentration of ethylene glycol monoallyl ether added to the medium in the PHA production culture was 1.0 g/L. Regarding the obtained PHA, the copolymerization ratio of the monomer units and the molecular weight were analyzed. The results are shown in Table 1.

### Comparative Example 1

### Production of PHA by KNK-005 trc-phaJ4b/ΔphaZ1,2,6 Strain in Medium Containing No Specific Alcohol

The KNK-005 trc-phaJ4b/ΔphaZ1,2,6 strain was cultured and the resultant was purified in the same manner as in Example 1 except that 2-propen-1-ol was not added to the medium in the PHA production culture, thereby obtaining a purified PHA. Regarding the obtained PHA, the copolymerization ratio of the monomer units and the molecular weight were analyzed. The results are shown in Table 1.

### Comparative Example 2

### Production of PHA by KNK-005 trc-phaJ4b/ΔphaZ1,2,6 Strain in Medium Containing 0.2 g/L of 2-Aminoethanol Hydrochloride

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, 2-aminoethanol hydrochloride was added to the medium so as to have a final concentration of 0.2 g/L. Regarding the obtained PHA, the copolymerization ratio of the monomer units and the molecular weight were analyzed. The results are shown in Table 1.

### Comparative Example 3

### Production of PHA by KNK-005 trc-phaJ4b/ΔphaZ1,2,6 Strain in Medium Containing 1.0 g/L of 2-Aminoethanol Hydrochloride

The KNK-005 trc-phaJ4b/ΔphaZ1,2,6 strain was cultured and the resultant was purified in the same manner as in Comparative Example 2 to obtain a purified PHA. However, the final concentration of 2 -aminoethanol hydrochloride added to the medium in the PHA production culture was 1.0 g/L. Regarding the obtained PHA, the copolymerization ratio of the monomer units and the molecular weight were analyzed. The results are shown in Table 1.

### Comparative Example 4

### Production of PHA by KNK-005 trc-phaJ4b/ΔphaZ1,2,6 Strain in Medium Containing 0.2 g/L of 3-Aminopropanol Hydrochloride

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, 2-aminopropanol hydrochloride was added to the medium so as to have a final concentration of 0.2 g/L. Regarding the obtained PHA, the copolymerization ratio of the monomer units and the molecular weight were analyzed. The results are shown in Table 1.

### Comparative Example 5

### Production of PHA by KNK-005 trc-phaJ4b/ΔphaZ1,2,6 Strain in Medium Containing 1.0 g/L of 2-Aminoethanol Hydrochloride

The KNK-005 trc-phaJ4b/ΔphaZ1,2,6 strain was cultured and the resultant was purified in the same manner as in Comparative Example 4 to obtain a purified PHA. However, the final concentration of 3-aminopropanol hydrochloride added to the medium in the PHA production culture was 1.0 g/L. Regarding the obtained PHA, the copolymerization ratio of the monomer units and the molecular weight were analyzed. The results are shown in Table 1.

### Comparative Example 6

### Production of PHA by KNK-005 trc-phaJ4b/ΔphaZ1,2,6 Strain in Medium Containing 0.2 g/L of 1,2-Ethanedithiol

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, 1,2-ethanedithiol was added to the medium so as to have a final concentration of 0.2 g/L. Regarding the obtained PHA, the copolymerization ratio of the monomer units and the molecular weight were analyzed. The results are shown in Table 1.

### Comparative Example 7

### Production of PHA by KNK-005 trc-phaJ4b/ΔphaZ1,2,6 Strain in Medium Containing 0.2 g/L of 1,3-Propanedithiol

The KNK-005 trc-phaJ4b/ΔphaZ1,2,6 strain was cultured and the resultant was purified in the same manner as in Example 1 to obtain a purified PHA. However, in the PHA production culture, in place of 2-propen-1-ol, 1,3-Propanedithiol was added to the medium so as to have a final concentration of 0.2 g/L. Regarding the obtained PHA, the copolymerization ratio of the monomer units and the molecular weight were analyzed. The results are shown in Table 1.

TABLE 1

| | | | | Copolymerization | | Molecular | |
|---|---|---|---|---|---|---|---|
| | Origin | Medium Addition Component | | Ratio | | Weight | |
| | of PHA | | Concentration | (mol %) | | (×10⁴) | |
| | Synthase | Type | (g/L) | 3HB | 3HHx | M_{w} | Mₙ |
| Example 1 | Aeromonas | 2-Propen-1-ol | 0.2 | 90.2 | 9.8 | 20 | 8.1 |
| Example 2 | | | 1 | 92.3 | 7.7 | 7.3 | 2.8 |
| Example 3 | | 3-Buten-1-ol | 0.2 | 89.4 | 10.6 | 135 | 46 |
| Example 4 | | | 1 | 90.7 | 9.3 | 28 | 10 |
| Example 5 | | 5-Hexen-1-ol | 0.2 | 90.5 | 9.5 | 252 | 78 |
| Example 6 | | | 1 | 88.9 | 11.1 | 53 | 15 |
| Example 7 | | 7-Propyn-1-ol | 0.2 | 91.4 | 8.6 | 6.4 | 2.7 |
| Example 8 | | | 1 | 94.3 | 5.7 | 2.5 | 1.2 |
| Example 9 | | 3-Butyn-1-ol | 0.2 | 89.7 | 10.3 | 51 | 20 |
| Example 10 | | | 1 | 89.3 | 10.7 | 13 | 5.0 |
| Example 11 | | 5-Hexyn-1-ol | 0.2 | 88.9 | 11.1 | 92 | 30 |
| Example 12 | | | 1 | 87.1 | 12.9 | 16 | 7 |
| Example 13 | | 2-Mercaptoethanol | 0.2 | 89.2 | 10.8 | 31 | 11 |
| Example 14 | | 3-Mercaptopropanol | 0.2 | 89.4 | 10.6 | 60 | 26 |
| Example 15 | | Ethylene Glycol | 0.2 | 89.7 | 10.3 | 124 | 32 |
| | | Monoallyl Ether | | | | | |
| Example 16 | | Ethylene Glycol | 1 | 90.2 | 9.8 | 39 | 13 |
| | | Monoallyl Ether | | | | | |
| Comparative Example 1 | | — | — | 88.9 | 11.1 | 279 | 128 |
| Comparative Example 2 | | 2-Aminoethanol | 0.2 | 89.8 | 10.2 | 398 | 206 |
| Comparative Example 3 | | Hydrochloride | 1 | 90.6 | 9.4 | 364 | 88 |
| Comparative Example 4 | | 3-Aminoethanol | 0.2 | 89.5 | 10.5 | 423 | 248 |
| Comparative Example 5 | | Hydrochrolide | 1 | 89.1 | 10.9 | 279 | 130 |
| Comparative Example 6 | | 1,2-Ethanedithiol | 0.2 | 90.7 | 9.3 | 325 | 87 |
| Comparative Example 7 | | 1,3-Propanedithiol | 0.2 | 90.4 | 9.6 | 239 | 91 |

### Results and Discussion

From the results in Table 1, it can be surmised as follows: in Examples 1 to 16 in which the PHA-producing microorganisms were cultured by adding an alcohol having an alkynyl group, an alkenyl group, a thiol group or an allyl group to the medium, the molecular weights (both the weight average molecular weight (M_{w}) and the number average molecular weight (Mₙ)) of the obtained PHA were decreased as compared to Comparative Example 1 in which the alcohol was not added, and thus the added alcohol functioned as a terminator; as a result, a PHA having an alkenyl group, an alkynyl group, a thiol group or an allyl group introduced at the terminal carboxy group was produced. However, when 5-hexen-1-ol was added, a significant decrease in molecular weight of the PHA was observed only when the addition concentration of 5-hexen-1-ol to the medium was 1 g/L.

FIGS. 1 and 2 are ¹H-NMR charts of Examples 8 and 16, respectively. For comparison, FIG. 3 shows a ¹H-NMR chart of Comparative Example 1. In FIG. 1, a peak attributable to methylene proton in a propynyl group appears in the vicinity of 4.7 ppm, as compared to FIG. 3. In FIG. 2, peaks attributable to ally groups appear in the vicinity of 4.5 ppm and 5.9 ppm. This indicates that these groups were introduced.

On the other hand, in Comparative Examples 2 to 5, even when 2-aminoethanol or 3-amino-1-propanol was added to the medium, no decrease in the molecular weight of PHA was observed. This result seems to be caused by the fact that these amino alcohols were metabolized by microorganisms. Further, in Comparative Examples 6 and 7, it is considered that even when 1,2-ethanedithiol and 1,3-propanedithiol are added to the medium, no decrease in the molecular weight of PHA is observed and dithiol does not function as the terminator.

The above results suggested that the microorganism having the PHA synthase gene derived from the genus Aeromonas is cultured by adding an alcohol having an alkynyl group, an alkenyl group, a thiol group or an allyl group to the medium, thereby producing a PHA having an alkenyl group, an alkynyl group, a thiol group or an allyl group introduced at the terminal carboxy group.

### Production Example 1

### Preparation of H16 ΔphaZ1,2,6 Strain

In order to prepare a H16ΔphaZ1,2,6 strain, first, based on the KNK-005 ΔphaZ1,2,6 strain described in WO2014/065253, the H16 ΔphaC1 ΔphaZ1,2,6 strain in which the PHA synthase gene was disrupted was prepared by the following procedure. The KNK-005 ΔphaZ1,2,6 strain is a strain in which the full-lengths of the phaZ1 and phaZ6 genes on the chromosome are deleted, the 16th codon to stop codon of the phaZ2 gene are deleted, and a PHA synthase gene represented by SEQ ID NO: 5 is present on the chromosome.

First, a plasmid for deleting the full-length PHA synthase gene of the KNK-005 ΔphaZ1,2,6 strain was prepared. The genomic DNA of C. necator H16 strain as a template was subjected to PCR with DNAs respectively represented by SEQ ID NOs: 6 and 7 as primer pairs. KOD-plus (TOYOBO CO., LTD.) was used as the polymerase. Similarly, PCR was performed with DNAs respectively represented by SEQ ID NOs: 8 and 9 as primer pairs. Two kinds of the DNA fragments obtained by the PCR as templates were subjected to PCR with DNAs respectively represented by SEQ ID NOs: 6 and 9 as primer pairs under the same conditions, and the obtained DNA fragment was digested with restriction enzyme SmiI. This DNA fragment was ligated to a DNA fragment obtained by digesting the vector pNS2X-sacB described in JP 2007-259708 A with SmiI using a DNA ligase (Ligation High, TOYOBO CO., LTD.) to prepare a PHA synthase gene disrupting-plasmid pNS2X-sacB-ΔphaC1UL having a base sequence located upstream of the phaC1 gene and a base sequence located downstream of the phaC1 gene.

The PHA synthase gene disrupting-plasmid pNS2X-sacB-ΔphaC1UL was introduced into an Escherichia coli S17-1 strain (ATCC 47055), and the Escherichia coli S17-1 strain and the KNK-005 ΔphaZ1,2,6 strain were mix-cultured on Nutrient Agar medium (DIFCO) to be subjected to conjugal transfer.

From the strains after the conjugal transfer, strains growing on Simmons agar medium containing 250 mg/L of kanamycin sulfate (2 g/L of sodium citrate, 5 g/L of sodium chloride, 0.2 g/L of magnesium sulfate heptahydrate, 1 g/L of ammonium dihydrogen phosphate, 1 g/L of dipotassium hydrogen phosphate, 15 g/L of agar, pH 6.8) were selected, and a strain in which the plasmid had been integrated on the chromosome of the KNK-005 ΔphaZ1,2,6 strain was obtained. This strain was cultured for two generations in Nutrient Broth medium (DIFCO), and then strains growing on Nutrient Agar medium containing 15% sucrose were selected. Strains in which the full-length PHA synthase gene represented by SEQ ID NO: 5 on the chromosome had been deleted were selected by PCR from the obtained strains, and one strain was named H16 ΔphaC1 ΔphaZ1,2,6 strain. The H16 ΔphaC1 ΔphaZ1,2,6 strain is a strain in which the C. necator H16 strain is used as a parent strain, the full-lengths of phaZ1 and phaZ6 genes on the chromosome are deleted, the 16th codon to stop codon of the phaZ2 gene are deleted, and the full-length of the phaC1 gene is deleted.

Next, based on the obtained H16 ΔphaC1 ΔphaZ1,2,6 strain, a H16 ΔphaC1,2,6 strain in which the phaC1 gene derived from the C. necator H16 strain represented by SEQ ID NO: 10 had been inserted was prepared.

First, a plasmid for inserting the phaC1 gene of C. necator H16 strain on the chromosome was prepared. The genomic DNA of C. necator strain H16 as a template was subjected to PCR with DNAs respectively represented by SEQ ID NOs: 6 and 9 as primer pairs. KOD-plus was used as the polymerase. The obtained DNA fragment was digested with the restriction enzyme SmiI. This DNA fragment was ligated to a DNA fragment obtained by digesting pNS2X-sacB with SmiI using a DNA ligase to prepare a PHA synthase gene-disrupting plasmid pNS2X-sacB-phaC_{Re}+UL having a base sequence located upstream of the phaC1 gene, the phaC1 gene, and a base sequence located downstream of the phaC1 gene.

According to the same procedure as the PHA synthase gene disruption, a strain in which pNS2X-sacB-phaC_{Re}+UL had been integrated on the chromosome using H16 ΔphaC1 ΔphaZ1,2,6 strain as a parent strain was obtained. This strain was cultured for two generations in Nutrient Broth medium, and then strains growing on Nutrient Agar medium containing 15% sucrose were selected. Strains in which the phaC1 gene on the chromosome had been inserted were selected by PCR from the obtained strains, and one strain was named a H16 ΔphaZ1, 2, 6 strain. The H16 ΔphaZ1,2,6 strain is a strain in which the C. necator H16 strain is used as a parent strain, the full-lengths of the phaZ1 and phaZ6 genes on the chromosome are deleted, the 16th codon to stop codon of the phaZ2 gene are deleted, and a gene encoding a wild-type PHA synthase derived from Ralstonia eutropha is present on the chromosome.

### Production Example 2

### Preparation of ReSK003 Strain

An ReSK003 strain in which the PHA synthase gene consisting of the base sequence represented by SEQ ID NO: 11 had been inserted on the chromosome was prepared based on the H16 ΔphaC1 ΔphaZ1,2,6 strain described in Production Example 1. The PHA synthase gene consisting of the base sequence represented by SEQ ID NO: 11 is a gene encoding a PHA synthase consisting of the amino acid sequence represented by SEQ ID NO: 3 which is derived from Pseudomonas Sp. 61-3 and in which serine at position 325 is artificially replaced with threonine, serine at position 477 is artificially replaced with arginine, and glutamine at position 481 is artificially replaced with arginine.

First, a plasmid pCUP2-REP-phaC1_{Ps} expressing the PHA synthase gene derived from Pseudomonas Sp. 61-3 strain represented by SEQ ID NO: 13 was prepared in the presence of the expression regulatory sequence represented by SEQ ID NO: 12. The expression regulatory sequence represented by SEQ ID NO: 11 is the promoter of phaCAB operon derived from C. necator H16 strain. The genomic DNA of C. necator H16 strain as a template was subjected to PCR with DNAs respectively represented by SEQ ID NOs: 14 and 15 as primer pairs. KOD-plus was used as the polymerase. Similarly, the genomic DNA of Pseudomonas Sp. 61-3 strain as a template was subjected to PCR with DNAs respectively represented by SEQ ID NOs: 16 and 17 as primer pairs. Two kinds of the DNA fragments obtained by the PCR as templates were subjected to PCR with DNAs respectively represented by SEQ ID NOs: 14 and 17 as primer pairs under the same conditions, and the obtained DNA fragment was digested with restriction enzymes MunI and SpeI. This DNA fragment was ligated to a DNA fragment obtained by digesting the vector pCUP2 described in JP 2007-259708 A with MunI and SpeI using a DNA ligase to prepare the plasmid pCUP2-REP-phaC1_{Ps}.

Then, the pCUP2-REP-phaC1_{Ps} as a template was subjected to PCR with DNAs respectively represented by SEQ ID NOs: 14 and 18 as primer pairs. Similarly, PCR was performed using DNAs respectively represented by SEQ ID NOs: 19 and 20 as primer pairs. Similarly, PCR was performed using DNAs respectively represented by SEQ ID NOs: 21 and 17 as primer pairs. Three kinds of the DNA fragments obtained by the PCR as templates were subjected to PCR with DNAs respectively represented by SEQ ID NOs: 14 and 17 as primer pairs under the same conditions, and the obtained DNA fragment was digested with restriction enzymes MunI and SpeI. This DNA fragment was ligated to a DNA fragment obtained by digesting the vector pCUP2 described in JP 2007-259708 A with MunI and SpeI using a DNA ligase to prepare the plasmid pCUP2-REP-phaC1_{Ps}_{^{S325T,S477R,Q481R}}.

Then, a plasmid for inserting the PHA synthase gene represented by SEQ ID NO: 11 on the chromosome was prepared. The pCUP2-REP-phaC1_{Ps}_{^{S325T,S477R,Q481R}} as a template was subjected to PCR with DNAs respectively represented by SEQ ID NOs: 6 and 22 as primer pairs. KOD-plus was used as the polymerase. Similarly, PCR was performed using DNAs respectively represented by SEQ ID NOs: 23 and 9 as primer pairs. Two kinds of the DNA fragments obtained by the PCR as templates were subjected to PCR with DNAs respectively represented by SEQ ID NOs: 6 and 9 as primer pairs under the same conditions, and the obtained DNA fragment was digested with restriction enzyme SmiI. This DNA fragment was ligated to a DNA fragment obtained by digesting pNS2X-sacB with SmiI using a DNA ligase, thereby preparing a PHA synthase gene-disrupting plasmid pNS2X-sacB-ΔphaC1UL::STSRQR having a base sequence located upstream of the phaC1 gene, a PHA synthase gene represented by SEQ ID NO: 11, and a base sequence located downstream of the phaC1 gene.

According to the same procedure as in the PHA synthase gene insertion described in Production Example 1, the PHA synthase gene represented by SEQ ID NO: 11 was inserted on the chromosome using the H16 ΔphaC1 ΔphaZ1,2,6 strain as a parent strain and the pNS2X-sacB-ΔphaC1UL::STSRQR. The obtained strain was named ReSK003 strain. The ReSK003 strain is a strain in which a mutant PHA synthase gene derived from the genus Pseudomonas represented by SEQ ID NO: 11 is inserted on the chromosome using H16 ΔphaC1Δpha Z1,2,6 strain as a parent strain.

### Example 17

Production of PHA by H16 ΔphaZ1,2,6 Strain in Medium Containing 1.0 g/L of 2 -Propen-1-ol

The H16 ΔphaZ1,2,6 strain prepared in Production Example 1 was cultured and the resultant was purified in the same manner as in Example 1 to obtain a purified PHA. However, the final concentration of 2-propen-1-ol added to the medium was 1.0 g/L. Regarding the obtained PHA, the copolymerization ratio of the monomer units and the molecular weight were analyzed. The results are shown in Table 2.

### Example 18

Production of PHA by H16 ΔphaZ1,2,6 Strain in Medium Containing 1.0 g/L of 2-Propyn-1-ol

The H16 ΔphaZ1,2,6 strain prepared in Production Example 1 was cultured and the resultant was purified in the same manner as in Example 15 to obtain a purified PHA. However, in the PHA production culture, in place of 2-propen-1-ol, 2-propyn-1-ol was added to the medium so as to have a final concentration of 1.0 g/L. Regarding the obtained PHA, the copolymerization ratio of the monomer units and the molecular weight were analyzed. The results are shown in Table 2.

### Comparative Example 8

### Production of PHA by H16 ΔphaZ1,2,6 Strain in Medium Not Containing Specific Alcohol

The H16 ΔphaZ1,2,6 strain prepared in Production Example 1 was cultured and the resultant was purified in the same manner as in Example 15 except that 2-propen-1-ol was not added to the medium in the PHA production culture, thereby obtaining a purified PHA. Regarding the obtained PHA, the copolymerization ratio of the monomer units and the molecular weight were analyzed. The results are shown in Table 2.

TABLE 2

| | | Medium | | | | Molecular | |
|---|---|---|---|---|---|---|---|
| | Origin | Addition Component | | Copolymerization | | Weight | |
| | of PHA | | Concentration | Ratio (mol %) | | (×10⁴) | |
| | Synthase | Type | (g/L) | 3HB | 3HHx | M_{w} | Mₙ |
| Example 17 | Ralstonia | 2-Propen-1-ol | 1.0 | 100 | 0.0 | 76 | 30 |
| Example 18 | eutropha | 2-Propyn-1-ol | 1.0 | 100 | 0.0 | 59 | 23 |

| | | | | | | |
|---|---|---|---|---|---|---|
| Comparative Example 8 | H16 | — | 100 | 0.0 | 243 | 93 |

### Results and Discussion

From the results in Table 2, it can be surmised as follows: in Examples 17 and 18 in which 2-propen-1-ol or 2-propyn-1-ol was added to the medium, the molecular weights (both M_{w} and Mₙ) of the obtained PHA were decreased as compared to Comparative Example 8 in which the alcohol was not added, and thus the added alcohol functioned as a terminator; as a result, a PHA having a propenyl group or a propynyl group introduced at the terminal carboxy group was produced. The above results suggested that the microorganism having the PHA synthase gene derived from the genus Ralstonia is cultured by adding an alcohol having an alkynyl group or an alkenyl group to the medium, thereby producing a PHA having an alkenyl group or an alkynyl group introduced at the terminal carboxy group.

### Example 19

Production of PHA by ReSK003 Strain in Medium Containing 1.0 g/L of 2-Propen-1-ol

The ReSK003 strain prepared in Production Example 2 was cultured and the resultant was purified in the same manner as in Example 1 to obtain a purified PHA. However, the final concentration of 2 -propen-1-ol added to the medium was 1.0 g/L. Regarding the obtained PHA, the copolymerization ratio of the monomer units and the molecular weight were analyzed. The results are shown in Table 3.

### Example 20

Production of PHA by ReSK003 Strain in Medium Containing 1.0 g/L of 2-Propyn-1-ol

The ReSK003 strain prepared in Production Example 1 was cultured and the resultant was purified in the same manner as in Example 19 to obtain a purified PHA. However, in the PHA production culture, in place of 2-propen-1-ol, 2-propyn-1-ol was added to the medium so as to have a final concentration of 1.0 g/L. Regarding the obtained PHA, the copolymerization ratio of the monomer units and the molecular weight were analyzed. The results are shown in Table 3.

### Comparative Example 9

### Production of PHA by ReSK003 Strain in Medium Not Containing Specific Alcohol

The ReSK003 strain prepared in Production Example 1 was cultured and the resultant was purified in the same manner as in Example 17 except that 2-propen-1-ol was not added to the medium in the PHA production culture, thereby obtaining a purified PHA. Regarding the obtained PHA, the copolymerization ratio of the monomer units and the molecular weight were analyzed. The results are shown in Table 3.

TABLE 3

| | | Medium | | | | Molecular | |
|---|---|---|---|---|---|---|---|
| | Origin | Addition Component | | Copolymerization | | Weight | |
| | of PHA | | Concentration | Ratio (mol %) | | (×10⁴) | |
| | Synthase | Type | (g/L) | 3HB | 3HHx | M_{w} | Mₙ |
| Example 19 | Pseudomonas | 2-Propen-1-ol | 1.0 | 96.4 | 3.6 | 20 | 9.1 |
| Example 20 | Sp. 61-3 | 2-Propyn-1-ol | 1.0 | 95.4 | 4.6 | 8.2 | 4.1 |

| | | | | | |
|---|---|---|---|---|---|
| Comparative Example 9 | — | 96.0 | 4.0 | 39 | 16 |

### Results and Discussion

From the results in Table 3, it can be surmised as follows: in Examples 19 and 20 in which 2-propen-1-ol or 2-propyn-1-ol was added to the medium, the molecular weights (both M_{w} and Mₙ) of the obtained PHA were decreased as compared to Comparative Example 9 in which the alcohol was not added, and thus the added alcohol functioned as a terminator; as a result, a PHA having a propenyl group or a propynyl group introduced at the terminal carboxy group was produced. The above results suggested that the microorganism having the PHA synthase gene derived from the genus Pseudomonas is cultured by adding an alcohol having an alkynyl group or an alkenyl group to the medium, thereby producing a PHA having an alkenyl group or an alkynyl group introduced at the terminal carboxy group.

1. A polyhydroxyalkanoic acid that is a homopolymer or copolymer of R-3-hydroxyalkanoic acid and has a repeating unit of the formula (1):
[—C*HR¹—CH₂—CO—O—] (1),
(where R¹ is an alkyl group represented by CₙH₂ₙ₊₁, n is an integer of 1 to 15, and * represents an asymmetric carbon,
wherein
(1) when the polyhydroxyalkanoic acid is a homopolymer of the R-3-hydroxyalkanoic acid,
an alkynyl group having 4 to 8 carbon atoms,
an alkenyl group having 3 to 8 carbon atoms,
a mercaptoalkyl group having 2, 4 to 8 carbon atoms,
an azidated alkyl group having 3 to 8 carbon atoms, or
an allyl (poly)oxyalkyl group in which an alkyl group has 2 to 6 carbon atoms, is bonded to a terminal carboxy group of the polyhydroxyalkanoic acid; and
(2) when the polyhydroxyalkanoic acid is a copolymer of the R-3-hydroxyalkanoic acid,
an alkynyl group having 3 to 8 carbon atoms,
an alkenyl group having 3 to 8 carbon atoms,
a mercaptoalkyl group having 2 to 8 carbon atoms,
an azidated alkyl group having 3 to 8 carbon atoms, or
an ally (poly)oxyalkyl group in which an alkyl group has 2 to 6 carbon atoms, is bonded to a terminal carboxy group of the polyhydroxyalkanoic acid.

2. A polyhydroxyalkanoic acid, having an alkyne group, an alkene group, a thiol group or an azide group introduced at a terminal carboxy group and produced by a microorganism,
wherein
the polyhydroxyalkanoic acid is composed of a plurality of monomers selected from the group consisting of 3-hydroxybutyric acid, 3-hydroxypropionic acid, 4-hydroxybutyric acid, 3-hydroxyvaleric acid, 5-hydroxyvaleric acid, 3-hydroxyhexanoic acid, 6-hydroxyhexanoic acid, 3-hydroxyheptanoic acid, 3-hydroxyoctanoic acid, 3-hydroxynonanoic acid, 3-hydroxydecanoic acid, 3-hydroxyundecanoic acid, and 3-hydroxydodecanoic acid.

3. A polyhydroxyalkanoic acid, having an alkyne group, an alkene group, a thiol group or an azide group introduced at a terminal carboxy group and produced by a microorganism,
wherein
the polyhydroxyalkanoic acid is composed of a single monomer selected from the group consisting of 3-hydroxybutyric acid, 3-hydroxypropionic acid, 4-hydroxybutyric acid, 3-hydroxyvaleric acid, 5-hydroxyvaleric acid, 3-hydroxyhexanoic acid, 6-hydroxyhexanoic acid, 3-hydroxyheptanoic acid, 3-hydroxyoctanoic acid, 3-hydroxynonanoic acid, 3-hydroxydecanoic acid, 3-hydroxyundecanoic acid, and 3-hydroxydodecanoic acid;
the alkyne group is a butynyl group, a pentynyl group or a hexynyl group;
the alkene group is a propenyl group, a butenyl group, a pentenyl group or a hexenyl group; and
the thiol group is a mercaptoethyl group, a mercaptobutyl group, a mercaptopentyl group or a mercaptohexyl group.

4. The polyhydroxyalkanoic acid according to claim 1, wherein the R-3-hydroxyalkanoic acid comprises 3-hydroxybutyric acid.

5. The polyhydroxyalkanoic acid according to claim 4, wherein the R-3-hydroxyalkanoic acid further comprises 3-hydroxyhexanoic acid.

6. A method for producing the polyhydroxyalkanoic acid according claim 1, the method comprising:
culturing a microorganism capable of producing a polyhydroxyalkanoic acid in the presence of an alcohol having an alkyne group, an alkene group, a thiol group, an azide group or an allyl group.

7. A method for producing a polyhydroxyalkanoic acid including an alkyne group, an alkene group, a thiol group, an azide group or an allyl group introduced at a terminal carboxy group, the method comprising:
culturing a microorganism belonging to the genus Cupriavidus capable of producing a polyhydroxyalkanoic acid in the presence of an alcohol having 2 to 8 carbon atoms and having an alkyne group, an alkene group, a thiol group, an azide group or an allyl group.

8. The method according to claim 6, wherein the alcohol is a primary alcohol.

9. The method according to claim 6, wherein the microorganism is a microorganism having a gene encoding a polyhydroxyalkanoate synthase derived from the genus Aeromonas, Ralstonia or Pseudomonas.

10. The production method according to claim 8, wherein the microorganism is a microorganism belonging to the genus Cupriavidus.

11. The production method according to claim 7, wherein the microorganism is a transformant of Cupriavidus necator.

12. The polyhydroxyalkanoic acid according to claim 1, wherein the alkyne group, the alkene group, the thiol group, the azide group or the allyl group at the terminus of the polyhydroxyalkanoic acid is chemically modified.

13. A molded article, comprising the polyhydroxyalkanoic acid according to claim 1.

14. The polyhydroxyalkanoic acid according to claim 1, wherein a side chain of the polyhydroxyalkanoic acid does not have a functional group.
